(19) United States
(12) Patent Application Publication
(10) Pub. No.: US 2009/0023694 A1
(43) Pub. Date: Jan. 22, 2009

(54) Contraceptive

(75) Inventors: Georg Schramm, Stolberg (DE); Christa Kneip, Aachen (DE); Johannes Schneider, Stolberg (DE)

Correspondence Address: NORRIS, MCLAUGHLIN & MARCUS, PA, 875 THIRD AVENUE, 18TH FLOOR, NEW YORK, NY, 10022, US

(73) Assignee: GRUNENTHAL GMBH, Aachen (DE)

(21) Appl. No.: 12/169,328

(22) Filed: Jul. 8, 2008

Related U.S. Application Data
(63) Continuation of application No. PCT/EP2007/000551, filed on Jan. 23, 2007.

(30) Foreign Application Priority Data
Jan. 24, 2006 (DE) 10 2006 003 509.7

(51) Int. Cl. A61K 31/565

(52) U.S. Cl. 514/170

(57) ABSTRACT

The present invention relates to an administration form for hormonal contraception consisting of a particular number of hormone-containing daily units, comprising a hormone combination consisting of at least one oestrogen selected from the group comprising ethinyl oestradiol (I) and oestradiol (II) as the oestrogen component, and at least one metabolite of chlormadinone acetate selected from the group comprising 3α-hydroxy-6-chloro-17α-acetoxy-4,6-pregnadien-20-one (3α-hydroxy-chlormadinone acetate), 3β-hydroxy-6-chloro-17α-acetoxy-4,6-pregnadien-2-one (3β-hydroxy-chlormadinone acetate), optionally mixed with chlormadinone acetate and/or 3α-hydroxy-17α-acetoxy-5β-pregnan-20-one and/or 3β-hydroxy-17α-acetoxy-5β-pregnan-20-one as the gestagen component for the uninterrupted, daily, oral administration of the hormone-containing daily units, optionally in combination with 7 to 3 hormone-free daily units.

This application is a continuation of PCT/EP2007/000551 filed Jan. 23, 2007, which claims priority to the German application 10 2006 003 509.7 filed Jan. 24, 2006.

The present invention relates to an administration form for hormonal contraception comprising of a particular number of hormone-containing daily units, comprising a hormone combination consisting of at least one oestrogen selected from the group comprising ethinyl oestradiol (I) and oestradiol (II) as the oestrogen component, and at least one metabolite of chlormadinone acetate selected from the group comprising 3α-hydroxy-6-chloro-17α-acetoxy-4,6-pregnadien-20-one (3α-hydroxy-chlormadinone acetate), 3β-hydroxy-6-chloro-17α-acetoxy-4,6-pregnadien-20-one (3β-hydroxy-chlormadinone acetate), optionally mixed with chlormadinone acetate and/or 3α-hydroxy-17α-acetoxy-5β-pregnan-20-one and/or 3β-hydroxy-17α-acetoxy-5β-pregnan-20-one as the gestagen component for the uninterrupted, daily, oral administration of the hormone-containing daily units, optionally in combination with 7 to 3 hormone-free daily units.

It is known from hormonal contraceptives containing a hormone combination that chlormadinone acetate is an effective gestagen component. It is also known that chlormadinone acetate metabolises, inter alia, into the following metabolites: 3α-hydroxy-6-chloro-17α-acetoxy-4,6-pregnadien-20-one (3α-hydroxy-chlormadinone acetate), 3β-hydroxy-6-chloro-17α-acetoxy-4,6-pregnadien-20-one (3β-hydroxy-chlormadinone acetate), 3α-hydroxy-17α-acetoxy-5β-pregnan-20-one or 3β-hydroxy-17α-acetoxy-5β-pregnan-20-one.

It has surprisingly been found that the metabolites 3α-hydroxy-chlormadinone acetate and/or 3β-hydroxy-chlormadinone acetate optionally mixed with chlormadinone acetate and/or 3α-hydroxy-17α-acetoxy-5β-pregnan-20-one and/or 3β-hydroxy-17α-acetoxy-5β-pregnan-20-one, are ideally suited for contraception as gestagen components in combination with an oestrogen component.

It was also found that the gestagen components mentioned above may have a positive effect on mood swings in women.

Since, during their menstruation cycle, many women suffer from mood swings with no noticeable outside influences or troubles or with no pain or disorders connected to the menstruation cycle, there is a need to at least alleviate said mood swings, which are regarded by women as a psychological drawback and affect their quality of life, and therefore to improve mood during the entire course of a menstruation cycle in such a way that their overall mood is brightened.

For understandable reasons, women who seek an effective level of contraception and suffer from mood swings of this type no longer want to have to take several preparations, but would like to only have to take one unit, preferably once daily. This object is also achieved by the contraceptive according to the invention.

The contraceptive according to the invention comprises of a particular number of hormone-containing daily units, with a hormone combination consisting of at least one oestrogen selected from the group comprising ethinyl oestradiol (I) and oestradiol (II) as the oestrogen component, and at least one metabolite of chlormadinone acetate selected from the group comprising 3α-hydroxy-6-chloro-17α-acetoxy-4,6-pregnadien-20-one (3α-hydroxy-chlormadinone acetate), 3β-hydroxy-6-chloro-17α-acetoxy-4,6-pregnadien-20-one (3β-hydroxy-chlormadinone acetate), optionally mixed with chlormadinone acetate and/or 3α-hydroxy-17α-acetoxy-5-pregnan-20-one and/or 3-hydroxy-17α-acetoxy-5-pregnan-20-one as the gestagen component for the uninterrupted, daily, oral administration of the hormone-containing daily units, optionally in combination with 7 to 3 hormone-free daily units.

The used gestagen component according to the invention is one of the following components a) to f)

a) 3α-hydroxy-chlormadinone acetate or
b) 3β-hydroxy-chlormadinone acetate or
c) a mixture of a) and b) at any mixing ratio or
d) a mixture of a) and/or b) with up to 20% by weight, based on the total mixture of 3α-hydroxy-17α-acetoxy-5-pregnan-20-one and/or 3β-hydroxy-17α-acetoxy-5-pregnan-20-one, where the latter metabolites may be present at any mixing ratio, or
e) a mixture of chlormadinone acetate with a), b) or c) at a mixing ratio of from 10 to 90% by weight chlormadinone acetate and from 90 to 10% by weight of a) and/or b), based on the total mixture, or
f) a mixture of chlormadinone acetate with c) at a mixing ratio of from 10 to 90% by weight chlormadinone acetate and from 90 to 10% by weight of c), based on the total mixture and up to 20% by weight based on the total mixture, 3α-hydroxy-17α-acetoxy-5β-pregnan-20-one and/or 3β-hydroxy-17α-acetoxy-5β-pregnan-20-one, where the total mixture must always equal 100% by weight.

Daily unit according to the invention preferably contains a hormone combination comprising between 5 and 50 μg ethinyl oestradiol and/or between 0.5 and 4 mg oestradiol and between 1 and 10 mg of one of the above-mentioned gestagen components a) to f) and optionally conventional additives.

A hormone combination comprising of at least 15 μg ethinyl oestradiol and/or at least 0.5 mg oestradiol and at least 1 mg of the above-mentioned gestagen components a) to f) is further preferred in daily unit.

Particularly preferred are daily units which each consist of a hormone combination comprising at least 15 μg, preferably 20 μg or 30 μg ethinyl oestradiol and/or at least 0.5 mg, preferably 1 mg or 2 mg oestradiol, and at least 1 mg, preferably 2, 3, 4 or 5 mg of one of the gestagen components a) to f) and, optionally, conventional additives.

Due to the excellent contraceptive effect, a hormone combination of 20 μg ethinyl oestradiol and/or 1 mg oestradiol and ≧2 mg of the gestagen components a) to f) are more particularly preferred for preparing daily unit of the administration form according to the invention.

The contraceptive according to the invention is preferably formulated as tablets which also optionally contain conventional additives as well as the hormone combination mentioned.

These tablets are, in particular, provided in the form of at least 21, preferably 21 to 25, hormone combination-containing daily units which are intended for uninterrupted, oral administration followed by a 3 to 7-day break in administration or in combination with 7 to 3 hormone-free daily units for uninterrupted, oral administration by women.

In order to avoid bleeding, such as mid-cycle bleeding or withdrawal bleeding, as much as possible, the contraceptive according to the invention may also be provided in the form of hormone combination-containing daily units for uninterrupted administration over several years, preferably up to two years, particularly preferably up to one year, optionally in combination with 7 to 3 hormone-free daily units for uninterrupted administration or directly followed by a 7 to 3-day break in administration.

However, the contraceptive according to the invention may also be provided in an administration form with less than 365 hormone combination-containing daily units, such as between 77 and 193 or between 42 and 52 hormone combination-containing daily units for uninterrupted oral administration, followed by a break in administration over 7 to 3 days or in combination with 7 to 3 hormone-free daily units for uninterrupted administration.

As has already been mentioned, a correspondingly long break in administration may be made instead of the 7 to 3 hormone-free daily units. The oral administration form with the above-mentioned number of hormone combination-containing daily units may also accordingly be provided as a kit which comprises a plurality of said administration forms for continued administration, uninterrupted by a corresponding break in administration. Of course, a kit may also comprise a plurality of oral administration forms which provides for uninterrupted administration of the hormone combination-containing daily units in combination with the aforementioned number of hormone-free daily units for uninterrupted administration.

Each of the hormone combination-containing daily units preferably comprises the same amount of the oestrogen component and the gestagen component, i.e. both the amount of ethinyl oestradiol and/or oestradiol and the amount of one of the gestagen components a) to f) remains constant over an administration cycle which, as mentioned above, may last up to several years.

In a further embodiment, in order to achieve a contraceptive effect, the content of ethinyl oestradiol or oestradiol or the gestagen components a) to f) contained in the hormone combination-containing daily units may vary in a known manner according to a two-phase or three-phase administration cycle over 21 to 25 days.

For this purpose, the hormone combination-containing daily units preferably comprise 20 μg or less ethinyl oestradiol, preferably 20 μg or 30 μg ethinyl oestradiol or 1 mg oestradiol over all phases and a different phase-dependent amount of between 2 and 5 mg of one of the gestagen components a) to f).

In the case of a two-phase contraceptive, the administration cycle preferably begins with daily administration of daily unit containing between 2 and 3 mg of one of the gestagen components a) to f) in addition to ethinyl oestradiol or oestradiol as the gestagen component over a period of between 7 and 12 days, followed by daily administration of daily unit containing between 3 and 4 mg of the same gestagen component over a period of between 9 and 18 days, the amount of the gestagen component in the first phase always being lower than that in the second phase, but remaining constant in each case and the amount of ethinyl oestradiol or oestradiol per daily unit remaining constant and unchanged over both phases at 20 μg or 30 μg and 1 mg respectively.

If the contraceptive according to the invention is administered as a three-phase contraceptive, the administration cycle preferably begins with uninterrupted daily administration of daily unit containing between 2 and 3 mg of one of the gestagen components a) to f) in addition to ethinyl oestradiol or oestradiol as the oestrogen component over a period of from 6 to 7 days, followed by daily administration of daily unit containing 3 mg of one of the specified gestagen components in addition to ethinyl oestradiol or oestradiol over uninterrupted period of between 5 and 9 days and ends with daily uninterrupted administration of daily unit containing between 3 and 5 mg of one of the specified gestagen components over a period of between 5 and 14 days. In the case of a three-phase contraceptive, the daily units also contain different respective amounts of the identical gestagen components from phase to phase, where from the first to the third phase, the amount of gestagen components per daily unit increases, but remains constant within a phase, and the amounts of ethinyl oestradiol or oestradiol also remain constant over all phases in an identical amount per daily unit.

The hormone-containing daily units preferably each contain between 20 and 50 μg, particularly preferably 30 μg, even more particularly preferably 20 μg ethinyl oestradiol and/or preferably 1 to 4 mg, preferably 2 mg, particularly preferably 1 mg oestradiol as the oestrogen component both in the case of a pharmaceutical composition which provides what is known as a two-phase contraceptive effect and in the case of a three-phase contraceptive effect. In addition, in order to achieve a maximum level of reliability of the contraceptive effect, it is particularly important for each administration cycle to contain uninterrupted successive administration of between 21 and 25 hormone combination-containing daily units. Once the hormone combination-containing daily units have been administered, uninterrupted administration over 7 to 3 days of hormone-free daily units or a corresponding break in administration of the same length may follow directly.

By administering the contraceptive according to the invention, not only is an excellent contraceptive effect achieved, but, surprisingly, women who suffer from mood swings dependent on the menstruation cycle no longer have to alleviate or even prevent these, since not only is a deterioration in the state of mind to an emotional low point in such women prevented, but also the emotional condition, i.e. the state of mind of a woman during her entire menstruation cycle, is improved in such a way that, overall, mood is brightened. In particular, this effect is to be achieved with mono-phase administration.

The contraceptive according to the invention is preferably available as an oral administration form, particularly preferably in the form of tablets. In this case, one daily unit corresponds to one tablet. The tablets are preferably packed in blister packs corresponding to an administration cycle, preferably marked by the respective daily unit to be administered, and are commercially available as a pack containing at least one such blister pack, preferably at least three blister packs, for the respective number of administration cycles or for an intended uninterrupted administration.

### In Vitro Data

### 1) Determination of the Affinity of 3-α-OH-CMA and 3-β-OH-CMA to the Human Progesterone Receptor

The following test for determining the affinity of 3-α-OH-CMA and 3-β-OH-CMA to the human progesterone receptor is based on the publication of the experimental specifications of Eckert et al. (Cancer Research, 1982, V42, p. 139-144). The corresponding disclosure is included herein as a reference and is part of the disclosure of the present application.

The cytosolic fractions of MCF-7 cells, which contain the human progesterone receptor, were used. 2 nM [³H] R 5020 was used as a reference substance and 1 μM R 5020 was used to determine the nonspecific binding. The substances ([³H] R 5020, 3-α-H-CMA and 3-β-OH-CMA) were each incubated at the receptor for 20 hours at a temperature of approximately 4° C. Stock solutions (5×10⁻² M) of 3-α-OH-CMA and 3-β-OH-CMA were each pre-diluted in 75% DMSO at a ratio of 1:10 and were subsequently further diluted in 25% DMSO at a ratio of 1:5. The final concentrations of 3-α-OH-CMA and 3-β-OH-CMA in the test were 3×10¹⁰ M, 3×10⁻⁹ M, 1×10⁻⁸ M, 3×10⁻⁸ M, 1×10⁻⁷ M, 3×10⁻⁷ M, 1×10⁻⁶ M and 1×10⁵ M. After the aforementioned incubation period of the substances, the incubation batches were filtered and washed according to standard specifications and the radioactivity of the filter was determined using a scintillation measuring device. Standard specifications of this type are known to the person skilled in the art. The experiments were each carried out twice.

The corresponding IC₅₀ values were calculated via a non-linear regression analysis of the displacement curves using the Hill curve-fitting formula. A calculation method of this type is known to the person skilled in the art.

The corresponding Kᵢ values (inhibition constants) were determined using the Cheng-Prusoff-equation (Kᵢ=IC₅₀/(1+(L/K_{D})), where L corresponds to the concentration of the radioligand in the test and K_{D} corresponds to the affinity of the radioligand to the receptor.

The measured values are summarised in the table below.

### 2) Determination of the Affinity of 3-α-OH-CMA and 3-β-OH-CMA to the Human Androgen Receptor

The following test for determining the affinity of 3-α-OH-CMA and 3-β-OH-CMA to the human androgen receptor is based on the publication of the experimental specifications of Zava et al. (Endocrinology, 1979, V104, p. 1007-1012). The corresponding disclosure is included herein as a reference and is part of the disclosure of the present application.

The cytosolic fractions of LNCaP cells, which contain the human androgen receptor, were used. 0.5 nM [³H] methyltrienolone was used as a reference substance and 1 μM miboleron was used to determine the nonspecific binding. The substances ([³H] methyltrienolone, 3-α-OH-CMA and 3-β-OH-CMA) were each incubated at the receptor for 24 hours at a temperature of approximately 4° C. The stock solutions, the dilution series of 3-α-OH-CMA and 3-β-OH-CMA, the washing steps, the determination of radioactivity and the methods for calculating the respective IC₅₀ and Kᵢ values correspond to the experimental protocol described under point 1).

The measured values are summarised in the table below.

| | | |
|---|---|---|
| Substance | IC₅₀ [nM] | Kᵢ [nm] |

(human) progesterone receptor

| | | |
|---|---|---|
| 3-α-OH-CMA | 39 | 13 |
| 3-β-OH-CMA | 18 | 6 |

(human) androgen receptor

| | | |
|---|---|---|
| 3-α-OH-CMA | 100 | 83 |
| 3-β-OH-CMA | 25 | 20 |

The data shows that both 3-α-OH-CMA and 3-β-OH-CMA have a high affinity to the human progesterone receptor and to the human androgen receptor. Their respective contraceptive and anti-androgynous effects can thus be deduced.

It is known that endogenously formed neurosteroid allopregnanolone has a positive effect on mood. Plasma concentrations of allopregnanolone are reduced in female patients with depression. The positive effect of allopregnanolone on mood is attributed to its interaction with GABAA receptors. Allopregnanolone acts on said receptor of the central nervous system as a positive allosteric modulator and thus leads to anxiolytic and mood-brightening effects.

It has been found that 3-α-OH-CMA or 3-β-OH-CMA exhibit binding properties to GABAA, which are very similar to those of allopregnanolone. Allopregnanolone influences the binding of radioactively labelled muscimol (an agonist) to these receptors.

### 3) Influence of Allopregnanolone, 3-α-OH-CMA and 3-β-OH-CMA on the Binding of Muscimol to GABAA Receptors (Rat Brain)

The following tests for determining the influence of allopregnanolone, 3-α-OH-CMA and 3-β-OH-CMA on the binding of muscimol to GABAA receptors (in rats) are based on the publication of the experimental specifications of Snodgrass, S. R. (Nature, 1979, V273, p. 392-394). The corresponding disclosure is included herein as a reference and is part of the disclosure of the present application.

### A) Allopregnanolone

Membrane preparations of cerebral rat cortex material were prepared. 5 nM [³H] muscimol was used as a reference substance and 10 μM muscimol was used to determine the nonspecific binding. The substances ([³H] muscimol, allopregnanolone) were each incubated at the receptor for 10 minutes at a temperature of approximately 4° C. The stock solution (5×10⁻² M) of allopregnanolone was prediluted in 75% DMSO at a ratio of 1:10 and was subsequently further diluted in 25% DMSO at a ratio of 1:5. The final concentration of allopregnanolone in the test was 1×10⁻¹⁰ M, 1×10⁻⁹ M, 1×10⁻⁸ M, 1×10⁻⁷ M, 1×10⁻⁶ M and 1×10⁻⁵ M respectively. The washing steps and the determination of radioactivity were carried out as previously described under point 1.

It can be shown that, at a concentration of 1 μM allopregnanolone, the binding of radioactively labelled muscimol (5 nM) is increased by 38%.

### B) 3-α-OH-CMA and 3-β-OH-CMA

The influence of 3-α-OH-CMA and 3-β-OH-CMA on the binding of muscimol to GABAA receptors (in rats) was determined in a similar manner to the method described previously. Instead of allopregnanolone, 3-α-OH-CMA or 3-β-OH-CMA were used.

It can be shown that, at a concentration of 0.1 μM 3-β-OH-CMA, binding of the radioactively labelled muscimol (5 nM) is increased by 31% and, at a concentration of 0.001 μM 3-α-OH-CMA, by 12%.

Thus, a mood-brightening effect of 3-α-OH-CMA and 3-β-OH-CMA may be deduced.

### EXAMPLES

### Example 1

| Composition | Per tablet | |
|---|---|---|
| Oestradiol | 1.00 | mg |
| 3α-hydroxychlormadinone acetate | 2.00 | mg |
| 3β-hydroxychlormadinone acetate | 2.00 | mg |
| Povidone K30 | 3.000 | mg |
| Lactose | 32.000 | mg |
| Maize starch | 0.500 | mg |
| Highly dispersed silicon dioxide | 0.500 | mg |

Oestradiol and povidone K30 (PVP) were dissolved in 600 ml ethanol. The gestagen components (particle size 90%<50 μm), lactose and maize starch were mixed in a mixer/granulator (Diosna P25) for 5 minutes and were subsequently soaked and mixed with the ethanolic solution containing the oestradiol. The moistened mass was pressed through a 3 mm sieve and dried in a vacuum drying chamber. The dried granulate was disagglomerated through a 0.6 mm sieve, mixed with highly dispersed silicon dioxide and pressed on a tablet press with 5 mm moulds to form tablets weighing 50 mg.

The tablets were coated with a methylhydroxypropylcellulose-based coating with the following composition (2 mg coating per tablet)

| | | |
|---|---|---|
| Methylhydroxypropylcellulose 6 mPa × s | 0.1351 | kg |
| Polyethylene glycol 6000 | 0.0395 | |
| Propylene glycol | 0.0054 | kg |
| Purified water | 1.6200 | kg |

These coated tablets were each packed in a blister pack to form a contraceptive with 24 hormone-containing daily units and 4 correspondingly composed, hormone-free, coated tablets.

### Example 2

| Composition | Per tablet | |
|---|---|---|
| Ethinyl oestradiol | 0.020 | mg |
| Chlormadinone acetate | 1.00 | mg |
| 3β-hydroxychlormadinone acetate | 2.00 | mg |
| 3α-hydroxy-17α-acetoxy-5β-pregnan-20-one | 1.00 | mg |
| Povidone K30 | 3.000 | mg |
| Lactose | 31.980 | mg |
| Maize starch | 0.500 | mg |
| Highly dispersed silicon dioxide | 0.500 | mg |

The tablets were prepared as disclosed in Example 1 and coated with a coating of the composition according to Example 1 (2 mg coating per tablet).

The coated tablets were packed in a blister pack to form a contraceptive with 24 hormone-containing daily units and 4 correspondingly composed, coated, hormone-free tablets.

1. Administration form for hormonal contraception comprising at least one hormone-containing daily units, comprising a hormone combination comprising at least one oestrogen selected from the group comprising ethinyl oestradiol (I) and oestradiol (II) as the oestrogen components, and at least one metabolite of chlormadinone acetate selected from the group comprising 3α-hydroxy-6-chloro-17α-acetoxy-4,6-pregnadien-20-one (3α-hydroxy-chlormadinone acetate), 3β-hydroxy-6-chloro-17α-acetoxy-4,6-pregnadien-20-one (3β-hydroxy-chlormadinone acetate), optionally mixed with chlormadinone acetate and/or 3α-hydroxy-17α-acetoxy-5β-pregnan-20-one and/or 3β-hydroxy-17α-acetoxy-5β-pregnan-20-one as the gestagen component for uninterrupted, daily, oral administration of the hormone-containing daily units, optionally in combination with 7 to 3 hormone-free daily units.

2. Administration form according to claim 1, wherein the hormone combination comprises of between 5 and 50 μg ethinyl oestradiol and/or between 0.5 and 4 mg oestradiol and between 1 and 10 mg gestagen component and optionally conventional additives.

3. Administration form according to claim 2, wherein the hormone combination comprises between 5 and 30 μg ethinyl oestradiol and/or between 0.5 and 2 mg oestradiol and between 1 and 10 mg gestagen component and optionally conventional additives.

4. Administration form according to claim 1, wherein the gestagen component comprises one of the following components
a) 3α-hydroxy-chlormadinone acetate or
b) 3β-hydroxy-chlormadinone acetate or
c) a mixture of a) and b) at any mixing ratio or
d) a mixture of a) and/or b) with up to 20% by weight, based on the total mixture, of 3α-hydroxy-17α-acetoxy-5β-pregnan-20-one and/or 3β hydroxy-17α-acetoxy-5β-pregnan-20-one, or
e) a mixture of chlormadinone acetate with a) and/or b) at a mixing ratio of from 10 to 90% by weight chlormadinone acetate and from 90 to 10% by weight of a) and/or b), based on the total mixture, or
f) a mixture of chlormadinone acetate with c) at a mixing ratio of from 10 to 90% by weight chlormadinone acetate and from 90 to 10% by weight of c), based on the total mixture and up to 20% by weight, based on the total mixture, of 3α-hydroxy-17α-acetoxy-5β-pregnan-20-one and/or 3β-hydroxy-17α-acetoxy-5β-pregnan-20-one, the total mixture always having to equal 100% by weight.

5. Administration form according to claim 1, wherein one daily unit comprises a hormone combination comprises in each daily unit 15 μg, 20 μg or 30 μg ethinyl oestradiol and/or 1 mg or 2 mg oestradiol and 1, 2, 3, 4 or 5 mg respectively of one of the gestagen components a) to f) and, optionally, conventional additives.

6. Administration form according to claim 5, wherein the hormone combination comprises in each daily unit of 20 μg ethinyl oestradiol and/or 1 mg oestradiol and ≧2 mg of the gestagen components a) to f).

7. Administration form according to claim 1, comprising at least 21 hormone-containing daily units and optionally 7 to 3 hormone-free daily units.

8. Administration form according to claim 7, wherein the maximum number of hormone-containing daily units corresponds to uninterrupted administration for several years, optionally in combination with 7 to 3 hormone-free daily units for uninterrupted administration for 7 to 3 days.

9. Administration form according to claim 8, comprising up to 730, hormone-containing daily units and optionally 7 to 3 hormone-free daily units.

10. Administration form according to claim 8, comprising between 77 and 193 hormone-containing daily units, optionally in combination with 7 to 3 hormone-free daily units.

11. Administration form according to claim 8, comprising between 42 and 52 hormone-containing daily units, optionally in combination with 7 to 3 hormone-free daily units.

12. Administration form according to claim 8, between 21 and 25 hormone-containing daily units, optionally in combination with 7 to 3 hormone-free daily units.

13. Administration form according to claim 1, wherein the hormone-containing daily units each comprise equal amounts of the oestrogen component the gestagen component.

14. Administration form according to claim 1, wherein the daily units are in the form of tablets.

15. Kit comprising at least one administration form for hormonal contraception according to of claim 1.

16. Kit according to claim 14, comprising a plurality of administration forms.

17. A method for monophase hormonal contraception comprising administering a hormone combination of ethinyl oestradiol and/or oestradiol as the oestrogen component and at least one metabolite of chlormadinone acetate selected from the group comprising 3α-hydroxy-6-chloro-17α-acetoxy-4,6-pregnadien-20-one (3α-hydroxy-chlormadinone acetate), 3β-hydroxy-6-chloro-17α-acetoxy-4,6-pregnadien-20-one (3β-hydroxy-chlormadinone acetate), optionally mixed with chlormadinone acetate and/or 3α-hydroxy-17α-acetoxy-5β-pregnan-20-one and/or 3β-hydroxy-17α-acetoxy-5β-pregnan-20-one as the gestagen component for at least 21 days, optionally in combination with 7 to 3 hormone-free daily units.

18. Administration form according to claim 6 wherein the hormone combination comprises in each daily unit 30 μg of ethinyl oestradiol.

19. Administration form according to claim 8 wherein the maximum number of hormone-containing daily units corresponds to uninterrupted administration for up to two years.

20. Administration form according to claim 19 wherein the maximum number of hormone-containing daily units corresponds to uninterrupted administration for up to one year.

21. Administration form according to claim 9 comprising up to 365 hormone-containing daily units and optionally 7 to 3 hormone-free daily units.
